(19) United States
(12) Patent Application Publication
(10) Pub. No.: US 2022/0041968 A1
(43) Pub. Date: Feb. 10, 2022

(54) CELL ADHESION COMPOSITION AND CELL ADHESION SUBSTRATE

(71) Applicant: HAMAMATSU PHOTONICS K.K., Hamamatsu-shi Shizuoka (JP)

(72) Inventors: Yuji KIMURA, Hamamatsu-shi Shizuoka (JP); Sayaka KAZAMI, Hamamatsu-shi Shizuoka (JP); Hiroyasu ITOH, Hamamatsu-shi Shizuoka (JP)

(73) Assignee: HAMAMATSU PHOTONICS K.K., Hamamatsu-shi Shizuoka (JP)

(21) Appl. No.: 17/310,783

(22) PCT Filed: Jan. 20, 2020
(86) PCT No.: PCT/JP2020/001755

§ 371 Date: Aug. 24, 2021

(30) Foreign Application Priority Data
Feb. 28, 2019 (JP) 2019-035675

(51) Int. Cl. C12M 1/00; C12N 1/02; C12M 1/12; C12M 3/06

(52) U.S. Cl. CPC C12M 23/20; C12M 23/16; C12M 25/00; C12N 1/02

(57) ABSTRACT

A cell adhesion composition according to one aspect of the present invention comprises: an amphiphilic compound; and a conjugate of a DNA and a hydrophilic molecule, wherein the amphiphilic compound has a hydrophobic group that can non-covalently bond to a cell membrane, and a hydrophilic group, and wherein a weight-average molecular weight of the hydrophilic molecule of the conjugate is larger than a weight-average molecular weight of a hydrophilic molecule from which the hydrophilic group of the amphiphilic compound derives. According to such a cell adhesion composition, it is possible to impart a cell adhesion ability to a base material at an arbitrary timing by using light having an arbitrary wavelength.

### TECHNICAL FIELD

The present invention relates to a cell adhesion composition and a cell adhesion base material.

### BACKGROUND ART

As a method of controlling cell adhesive properties of a base material by light, various techniques as disclosed in Patent Literature 1 to Patent Literature 3 are known. According to these techniques, it is possible to impart a cell adhesion ability to a base material by irradiating the base material with light.

### CITATION LIST

### Patent Literature

[Patent Literature 1] Japanese Unexamined Patent Publication No. 2015-73460
[Patent Literature 2] Japanese Unexamined Patent Publication No. 2009-65945
[Patent Literature 3] Japanese Unexamined Patent Publication No. 2006-8975

### SUMMARY OF INVENTION

### Technical Problem

In the techniques disclosed in Patent Literature 1 to Patent Literature 3, light used to impart a cell adhesion ability to a base material is limited to light having a specific wavelength such as ultraviolet rays (UV). UV is not preferable because it damages cells. In addition, since cells adhered to a base material are often observed using a fluorescent dye, when the wavelength of the light used to impart cell adhesion ability to a base material is limited to a specific wavelength, the choice of fluorescent dyes that can be used to observe cells is narrowed.

Accordingly, an object of the present invention is to impart a cell adhesion ability to a base material at an arbitrary timing by using light having an arbitrary wavelength.

### Solution to Problem

A cell adhesion composition according to one aspect of the present invention comprises: an amphiphilic compound; and a conjugate of a DNA and a hydrophilic molecule. The amphiphilic compound has a hydrophobic group that can non-covalently bond to a cell membrane, and a hydrophilic group. A weight-average molecular weight of the hydrophilic molecule of the conjugate is larger than a weight-average molecular weight of a hydrophilic molecule from which the hydrophilic group of the amphiphilic compound derives.

The hydrophilic group may be a residue of a hydrophilic molecule selected from the group consisting of polyalkylene glycol, polyglycerin, polysaccharide, polylactic acid, polyvinyl alcohol, polyacrylic acid, and polyacrylamide. The hydrophobic group may be an aliphatic hydrocarbon group having 7 to 22 carbon atoms, or a residue of a phospholipid having an aliphatic hydrocarbon group having 7 to 22 carbon atoms. The hydrophilic group is preferably a residue of polyethylene glycol. The hydrophobic group is preferably an aliphatic hydrocarbon group having 10 to 20 carbon atoms, or a residue of a phospholipid having an aliphatic hydrocarbon group having 10 to 20 carbon atoms. The hydrophilic molecule of the conjugate may be a hydrophilic molecule selected from the group consisting of polyalkylene glycol, polyglycerin, polysaccharide, polylactic acid, polyvinyl alcohol, polyacrylic acid, and polyacrylamide. The cell adhesion composition may comprise one or more conjugates per molecule of the amphiphilic compound. The weight-average molecular weight of the hydrophilic molecule of the conjugate may be more than 1 time the weight-average molecular weight of the hydrophilic molecule from which the hydrophilic group of the amphiphilic compound derives.

A cell adhesion base material according to one aspect of the present invention comprises: a base material; one or more amphiphilic compounds; and one or more conjugates of a DNA and a hydrophilic molecule. Each of the amphiphilic compounds has a hydrophobic group that can non-covalently bond to a cell membrane, and a hydrophilic group. The hydrophilic group of each of the amphiphilic compounds and the DNA of each of the conjugates are bound to the base material. A weight-average molecular weight of the hydrophilic molecule of the conjugate is larger than a weight-average molecular weight of a hydrophilic molecule from which the hydrophilic group of the amphiphilic compound derives.

The cell adhesion base material may comprise the one or more conjugates per molecule of the amphiphilic compound.

A cell adhesion base material according to another aspect of the present invention comprises: a base material; and one or more conjugates of an amphiphilic compound and a DNA. Each of amphiphilic compounds has a hydrophobic group that can non-covalently bond to a cell membrane, and a hydrophilic group bound to the DNA. The DNA is bound to the base material.

The cell adhesion base material may further comprise a photoreactive substance that produces active oxygen upon light irradiation.

A microchannel device according to one aspect of the present invention comprises a channel in which at least a part of an inner side is coated with the above-described cell adhesion composition.

The microchannel device may comprise: a first channel; a second channel adjacent to the first channel; and a communicating portion that connects the first channel to the second channel and has an opening on the side of the first channel in which a cell can be captured, and an inner side of the first channel may be coated with the above-described cell adhesion composition.

A method for adhering a cell onto a base material according to one aspect of the present invention comprises: coating the base material with the above-described cell adhesion composition; bringing a photoreactive substance that produces active oxygen upon light irradiation into contact with the base material; irradiating the base material with light to excite the photoreactive substance; and bringing the cell into contact with the base material.

### Advantageous Effects of Invention

According to the present invention, it is possible to impart a cell adhesion ability to a base material at an arbitrary timing by using light having an arbitrary wavelength, and a light irradiation time required for imparting the cell adhesion ability to the base material is short. More specifically, according to the present invention, a base material onto which an arbitrary cell can be adhered at an arbitrary timing by using light having an arbitrary wavelength, a microchannel device comprising this base material, and a composition that can be used to manufacture them are provided. Furthermore, according to the present invention, a method by which an arbitrary cell can be adhered to a base material at an arbitrary timing by using light having an arbitrary wavelength is provided. Furthermore, according to the present invention, it is possible to easily obtain a cell pattern in an arbitrary shape.

### BRIEF DESCRIPTION OF DRAWINGS

FIG. 1(A) and FIG. 1(B) are schematic views showing an example of a microchannel device.

FIG. 2(A), FIG. 2(B), and FIG. 2(C) are schematic views showing an outline of a method for adhering a cell onto a base material.

### DESCRIPTION OF EMBODIMENTS

A cell adhesion composition according to one embodiment of the present invention comprises an amphiphilic compound, and a conjugate of a DNA and a hydrophilic molecule. The amphiphilic compound has a hydrophobic group that can non-covalently bond to a cell membrane, and a hydrophilic group. When the cell adhesion composition is brought into contact with a base material, the hydrophilic group of the amphiphilic compound and the DNA of the conjugate are bound to the base material, and thereby the base material can be coated with the amphiphilic compound and the conjugate of a DNA and a hydrophilic molecule. From the viewpoint of improving the binding to the base material, a binding substance may be bound to the hydrophilic group of the amphiphilic compound and the DNA of the conjugate. The amphiphilic compound has a cell adhesion ability, whereas the conjugate of a DNA and a hydrophilic molecule has an action of masking the cell adhesion ability of the amphiphilic compound. Accordingly, the base material coated with the cell adhesion composition has a potential cell adhesion ability. As will be described later, by irradiating the base material with light to degrade the conjugate, the cell adhesion ability of the amphiphilic compound is exhibited, and thereby cells can be adhered to the base material.

The hydrophilic group may be a residue of one or more hydrophilic molecules selected from the group consisting of polyalkylene glycol, polyglycerin, polysaccharide, polylactic acid, polyvinyl alcohol, polyacrylic acid, and polyacrylamide. More specifically, the hydrophilic group may be a residue of one or more hydrophilic molecules selected from the group consisting of polyethylene glycol, polypropylene glycol, pentaerythritol, glycerin, diglycerin, triglycerin, tetraglycerin, pentaglycerin, hexaglycerin, heptaglycerin, and octaglycerin. The hydrophilic group is preferably a residue of polyethylene glycol. In the present specification, the residue of a hydrophilic molecule means a group obtained by removing one or more atoms (for example, hydrogen) or groups which are removed from the hydrophilic molecule when forming a covalent bond with another molecule.

From the viewpoint of enhancing the binding to the base material or to the binding substance, the hydrophilic group may have a reactive functional group. The reactive functional group is not particularly limited as long as it is a known reactive functional group, and it may be, for example, an N-hydroxysuccinimide (NHS) group or a maleimide group.

The hydrophilic group may be a residue of a hydrophilic molecule having a weight-average molecular weight of 200 or more, 400 or more, 600 or more, 1000 or more, 2000 or more, 3000 or more, 5000 or more, or 8000 or more. The hydrophilic group may be a residue of a hydrophilic molecule having a weight-average molecular weight of 20000 or less, 10000 or less, 8000 or less, 5000 or less, 3000 or less, 2000 or less, 1000 or less, or 600 or less. The weight-average molecular weight may be determined using, for example, gel permeation chromatography (GPC).

The hydrophobic group is not particularly limited as long as it can non-covalently bond to a cell membrane, and it may be, for example, an aliphatic hydrocarbon group having 7 to 22 carbon atoms, or a residue of a phospholipid having an aliphatic hydrocarbon group having 7 to 22 carbon atoms. The aliphatic hydrocarbon group may be saturated or unsaturated, and may be a straight chain or a branched chain. The aliphatic hydrocarbon group may have 10 to 20 or 11 to 18 carbon atoms. The aliphatic hydrocarbon group may be, for example, a saturated aliphatic hydrocarbon group such as an octyl group (C8), a decyl group (C10), a dodecyl group (C12), a tetradecyl group (C14), a hexadecyl group (C16), an octadecyl group (C18), an isostearyl group (C18), an eicosyl group (C20), and a docosyl group (C22); or may be, for example, an unsaturated aliphatic hydrocarbon group such as a myristoleyl group (C14), a palmitoleyl group (C16), an oleyl group (C18), a linoleyl group (C18), an arachidonyl group (C20), and an erucyl group (C22). The number of aliphatic hydrocarbon groups in the phospholipid may be 1 or more or 2 or more, and it is preferably 1 or 2. Examples of the phospholipid include phosphatidylethanolamine, phosphatidylglycerol, and phosphatidylserine. The phospholipid may be, for example, 1,2-distearoyl-sn-glycero-3-phosphoethanolamine (DSPE). Non-covalent bonds may be hydrophobic interactions. In the present specification, the residue of a phospholipid means a group obtained by removing one or more atoms (for example, hydrogen) or groups which are removed from the phospholipid when forming a covalent bond with another molecule.

The amphiphilic compound may specifically be, for example, a compound in which a hydrophilic molecule and a hydrophobic molecule are covalently bonded to each other, where the hydrophilic molecule is selected from the group consisting of polyalkylene glycol, polyglycerin, polysaccharide, polylactic acid, polyvinyl alcohol, polyacrylic acid, and polyacrylamide, and the hydrophobic molecule is selected from the group consisting of an aliphatic hydrocarbon having 7 to 22 carbon atoms and a phospholipid having an aliphatic hydrocarbon group having 7 to 22 carbon atoms. Details of the hydrophilic molecule and the hydrophobic molecule are as described above. The hydrophilic molecule may have the reactive functional groups described above. Specific examples of the amphiphilic compound include a compound (PEG-lipid) in which polyethylene glycol and an aliphatic hydrocarbon having 7 to 22 carbon atoms are covalently bonded to each other, and a compound (PEG-phospholipid) in which polyethylene glycol and a phospholipid having an aliphatic hydrocarbon group having 7 to 22 carbon atoms are covalently bonded to each other. The PEG-lipid may be, for example, oleyl-O-polyethylene glycol-succinyl-N-hydroxy-succinimidyl ester. The PEG-phospholipid may be, for example, N—[N′-(3′-maleimido-1′-oxopropyl)aminopropylpolyoxyethylene oxycarbonyl]-1,2-distearoyl-sn-glycero-3-phosphoethanolamine.

The DNA is not particularly limited as long as it can be degraded by active oxygen, and a DNA of any length and sequence may be used. For example, 17-mer to 30-mer, 18-mer to 25-mer, or 20-mer to 22-mer DNA is readily available. The DNA may be single-stranded or double-stranded. The DNA may have a reactive functional group from the viewpoint of enhancing the binding to the hydrophilic molecule and the base material or the binding molecule. The reactive functional group is not particularly limited, and for example, it may be selected from known reactive functional groups such as a carboxy group, a thiol group, and an amino group as appropriate, depending on the type of the hydrophilic molecule and base material or binding molecule. For example, if the binding molecule is bovine serum albumin (BSA) and the hydrophilic molecule is PEG having a maleimide group, the DNA may have a carboxy group that reacts with the amino group of BSA by a crosslinking agent, and a thiol group that reacts with the maleimide group of PEG

The hydrophilic molecule of the conjugate may be one or more hydrophilic molecules selected from the group consisting of polyalkylene glycol, polyglycerin, polysaccharide, polylactic acid, polyvinyl alcohol, polyacrylic acid, and polyacrylamide. More specifically, the hydrophilic molecule of the conjugate may be one or more hydrophilic molecules selected from the group consisting of polyethylene glycol, polypropylene glycol, pentaerythritol, glycerin, diglycerin, triglycerin, tetraglycerin, pentaglycerin, hexaglycerin, heptaglycerin, and octaglycerin. The hydrophilic molecule of the conjugate is preferably polyethylene glycol.

From the viewpoint of enhancing the binding to the DNA, the hydrophilic molecule of the conjugate may have a reactive functional group. The reactive functional group is not particularly limited, and it may be, for example, a known reactive functional group such as an NHS group and a maleimide group.

From the viewpoint of masking the cell adhesion ability of the amphiphilic compound, a weight-average molecular weight of the hydrophilic molecule may be, for example, 2000 or more, 5000 or more, or 10000 or more, and it may be 80000 or less, 60000 or less, 40000 or less, 30000 or less, 20000 or less, 10000 or less, or 5000 or less.

From the viewpoint of improving the binding to the base material, a binding substance may be conjugated to the hydrophilic group of the amphiphilic compound and the DNA of the conjugate. The binding substance is not particularly limited as long as it has a functional group capable of binding to the base material, the hydrophilic group of the amphiphilic compound, and the DNA of the conjugate. For example, the binding substance may be a protein such as BSA, ovalbumin, and collagen; or a polypeptide such as polylysine.

The cell adhesion composition may further comprise one or more photoreactive substances that produces active oxygen upon light irradiation. The photoreactive substance is not particularly limited as long as it is a substance that produces active oxygen upon light irradiation, and any photoreactive substance that can be excited by light having a desired wavelength may be selected. The photoreactive substance may be, for example, one or more photoreactive substances selected from the group consisting of a fluorescent dye, photosensitizer, and photocatalyst. The photoreactive substance is preferably a DNA-binding photoreactive substance capable of binding to a DNA, and is more preferably a DNA-binding fluorescent dye. A fluorescent dye may be, for example, a DNA-binding fluorescent dye selected from the group consisting of YOYO (registered trademark)-1, YO-PRO (registered trademark)-1, TOTO (registered trademark)-1, TO-PRO (registered trademark)-1, BOBO (registered trademark)-1, and BO-PRO (registered trademark)-1. Examples of the photosensitizer include porphyrin derivatives such as porfimer sodium and talaporfin sodium. Examples of photocatalyst include titanium (IV) oxide. From the viewpoint of preventing damage to cells, the photoreactive substance is preferably a substance excited by light of more than 380 nm. For example, the photoreactive substance may be a substance excited by light of 430 nm or more, 450 nm or more, or 480 nm or more.

The cell adhesion composition may include 1 or more, 5 or more, 10 or more, 15 or more, or 20 or more conjugates per molecule of the amphiphilic compound. A weight-average molecular weight of the hydrophilic molecule of the conjugate may be more than 1 time, 5 times or more, 10 times or more, or 20 times or more a weight-average molecular weight of the hydrophilic molecule from which the hydrophilic group of the amphiphilic compound derives. A combination of a weight-average molecular weight of the hydrophilic molecule from which the hydrophilic group of the amphiphilic compound derives and a weight-average molecular weight of the hydrophilic molecule of the conjugate may be, for example, 200 to 600 and 2000 to 5000, 1000 to 5000 and 10000 to 60000, or 8000 to 20000 and 10000 to 80000.

A cell adhesion base material according to one embodiment of the present invention comprises: a base material; one or more amphiphilic compounds, preferably a plurality of amphiphilic compounds; and one or more conjugates of a DNA and a hydrophilic molecule, preferably a plurality of the conjugates. At least a part of a surface of the base material is coated with the amphiphilic compounds and the conjugates, and the hydrophilic group of each of the amphiphilic compounds and the DNA of each of the conjugates are bound to the base material. Namely, the base material and the amphiphilic compound are bound such that each element is aligned in the order of base material-hydrophilic group-hydrophobic group, and the base material and the conjugate are bound such that each element is aligned in the order of base material-DNA-hydrophilic molecule. The cell adhesion base material according to the present embodiment may be obtained by coating the base material with the above-described cell adhesion composition. Details of the amphiphilic compound and the conjugate of a DNA and a hydrophilic molecule are as described above.

It is preferable that a material and a shape and a form of the base material be suitable for adhering cells, but they are not particularly limited. A material of the base material may be, for example, glass, ceramic, metal, or synthetic resin. The synthetic resin may be, for example, a polystyrene resin, a silicone resin, an acrylic resin, a polyethylene resin, a polypropylene resin, a polycarbonate resin, or an epoxy resin. The base material may have, for example, a shape and a form of a flat plate, a film, a particle, a rod, or a porous body. A surface of the base material may be a flat surface or a curved surface.

The base material may be a base material having a surface coated with a binding substance from the viewpoint of enhancing the binding to the hydrophilic group of the amphiphilic compound and to the DNA of the conjugate. Details of the binding substance are as described above.

The cell adhesion base material may further comprise a photoreactive substance that produces active oxygen upon light irradiation. Specifically, the photoreactive substance may be bound to the DNA of the conjugate. Details of the photoreactive substance are as described above.

The cell adhesion base material may comprise 1 or more, 5 or more, 10 or more, 15 or more, or 20 or more conjugates per molecule of the amphiphilic compound.

On the surface of the base material, the amphiphilic compounds are oriented such that hydrophilic groups are positioned on a side closer to the surface of the base material, and hydrophobic groups are positioned on a side farther from the surface of the base material; and the conjugates are oriented such that the DNA is positioned on a side closer to the surface of the base material, and hydrophilic molecules are positioned on a side farther from the surface of the base material. As described above, a weight-average molecular weight of the hydrophilic molecule of the conjugate is larger than a weight-average molecular weight of the hydrophilic molecule from which the hydrophilic group of the amphiphilic compound derives. Accordingly, the hydrophilic molecules of the conjugates are exposed on the outermost part of the cell adhesion base material according to the present embodiment, and the hydrophobic groups having the cell adhesion ability of the amphiphilic compounds are hidden under the hydrophilic molecules of the conjugates. As will be described later, by providing the photoreactive substance to the base material and then exciting them with light, the DNA of the conjugates is cleaved and the hydrophilic molecules are dissociated. Thereby, hydrophobic groups of the amphiphilic compounds are exposed to the outermost part. Therefore, according to the cell adhesion base material according to the present embodiment, it is possible to adhere arbitrary cells at an arbitrary timing by using light having an arbitrary wavelength. Furthermore, according to the cell adhesion base material according to the present embodiment, it is possible to easily obtain an arbitrary cell pattern.

A cell adhesion base material according to another embodiment of the present invention comprises: a base material; and one or more conjugates of an amphiphilic compound and a DNA, preferably a plurality of the conjugates. Each of the amphiphilic compounds has a hydrophobic group that can non-covalently bond to a cell membrane, and a hydrophilic group bound to the DNA. The DNA is bound to the base material. Namely, the base material and the conjugate are bound such that each element is aligned in the order of base material-DNA-hydrophilic group-hydrophobic group.

Details of the amphiphilic compounds, the DNA, and the base material are as described above. However, in the present embodiment, the amphiphilic compound is bound to the DNA instead of being bound to the base material or a binding substance. Furthermore, in the present embodiment, the DNA is bound to the hydrophilic group instead of being bound to the above-described hydrophilic molecule.

The DNA and the amphiphilic compound may be bound via a reactive functional group. The reactive functional group is not particularly limited, and it may be, for example, a known reactive functional group such as a carboxy group, a thiol group, an amino group, an NHS group, and a maleimide group.

The cell adhesion base material may further comprise a photoreactive substance that produces active oxygen upon light irradiation. Specifically, the photoreactive substance may be bound to the DNA of the conjugate. Details of the photoreactive substance are as described above.

On the surface of the base material, the conjugates of an amphiphilic compound and a DNA are oriented such that the DNA is positioned on a side closer to the surface of the base material, and hydrophobic groups are positioned on a side farther from the surface of the base material. Accordingly, the hydrophobic groups having the cell adhesion ability are exposed on the outermost part of the cell adhesion base material according to the present embodiment, and thereby cells are adhered. By providing the above-described photoreactive substance to the base material and then exciting them with light, the DNA is cleaved and the adhered cells are released from the base material together with the conjugates. Therefore, according to the cell adhesion base material according to the present embodiment, it is possible to release and recover arbitrary cells adhered to the base material at an arbitrary timing by using light having an arbitrary wavelength. Furthermore, according to the cell adhesion base material according to the present embodiment, it is possible to easily obtain an arbitrary cell pattern.

In one embodiment, the present invention provides a microchannel device comprising a channel in which at least a part of an inner side is coated with the above-described cell adhesion composition. The microchannel device is generally a device comprising one or more microchannels and can be used as a means for capturing and analyzing cells.

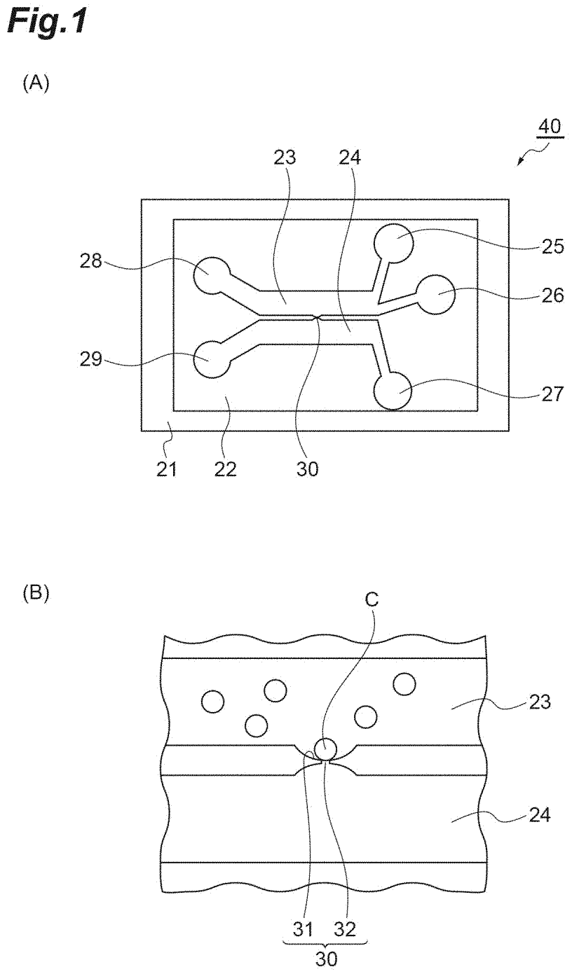

FIGS. 1(A) and 1(B) show an example of the microchannel device according to the present embodiment. A microchannel device 40 shown in FIG. 1(A) comprises a channel 23, a channel 24 adjacent to the channel 23, and a communicating portion 30 connecting the channel 23 to the channel 24. The channel 23, the channel 24, and the communicating portion 30 are all grooves provided on a substrate 22, and a cover glass 21 is laminated on the main surface on the side of the substrate 22 on which the grooves are formed. The substrate 22 is not particularly limited, and may be made of, for example, a resin such as silicone rubber (for example, dimethylpolysiloxane). When the substrate 22 is made of a resin, the channel 23, the channel 24, and the communicating portion 30 can be easily formed by photolithography.

Inlets 25 and 26, and an outlet 28 for a liquid are provided in the channel 23, and an inlet 27 and an outlet 29 for a liquid are provided in the channel 24. Liquid such as cell suspensions, samples, standard samples, and buffers, for example, are injected into the inlets 25 to 27. A liquid introduced from the inlets 25 and 26 into the channel 23 is discharged from the outlet 28 to the outside of the microchannel device 40, and a liquid introduced from the inlet 27 into the channel 24 is discharged from the outlet 29 to the outside of the microchannel device 40. The liquid may be injected into the inlets using, for example, a syringe. There may be as many inlets as the number of liquids used, but it is sufficient as long as there is at least one inlet for one channel. Therefore, the inlet 26 may not be provided, or one or more inlets may be added to the channel 23 and/or the channel 24. Similarly, one or more outlets may be added to the channel 23 and/or the channel 24.

FIG. 1(B) shows an enlarged view of the communicating portion 30. In this figure, a cell suspension is introduced into the channel 23. The communicating portion 30 comprises a hole 32 connecting the channel 23 to the channel 24 and an opening (open end) 31 in which a cell C can be captured. Here, “a cell C can be captured” means that the cell C present in the channel 23 can be held at the opening 31 on the channel 23 side under conditions in which the pressure in the channel 23 is higher than the pressure in the channel 24. In FIG. 1(B), the opening 31 forms a depression, but the shape of the opening 31 is not particularly limited as long as it can capture the cell C, and it may also be flat. The communicating portion 30 is required to have a shape through which the cell C cannot pass. Therefore, it is preferable that the hole diameter of the hole 32 be sufficiently smaller than the diameter of the cell C. Furthermore, in FIGS. 1(A) and 1(B), the communicating portion 30 connects the channel 23 to the channel 24 via the hole 32, but the hole 32 may be replaced with a slit. The opening 31 needs only be provided on the side of the channel in which the cell C is present. In FIG. 1(B), since the cell suspension is introduced into the channel 23, the opening 31 needs only be provided on the channel 23 side. In a case of introducing a cell suspension into the channel 24, the opening 31 needs only be provided on the channel 24 side.

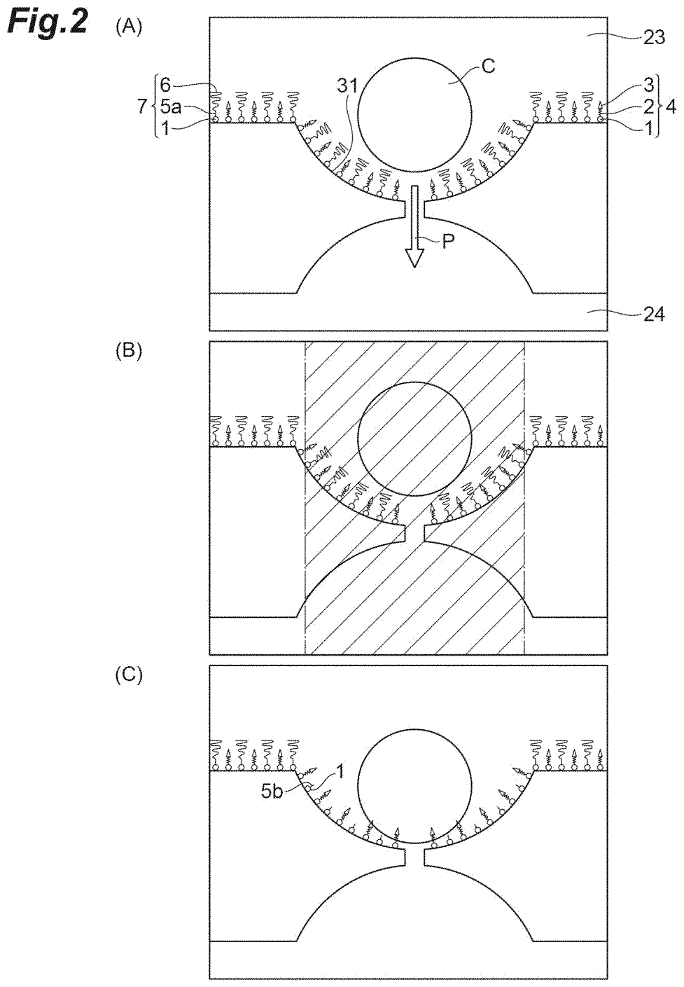

FIG. 2(A) shows a further enlarged schematic view of the communicating portion 30. In this figure, the cell C is captured at the opening 31 by a force acting in a direction from the channel 23 to the channel 24 (the direction indicated by an arrow P in the figure). The force acting in the direction of the arrow P is generated by a pressure difference between the channel 23 and the channel 24. In FIG. 2(A), the cell C is not adhered to an inner wall constituting the channel 23, and if a pressure difference between the channel 23 and the channel 24 is eliminated, the cell C is released from the opening 31.

An inner side of the channel 23 is coated with the above-described cell adhesion composition. In this figure, an amphiphilic compound 4 comprises a binding substance 1, a hydrophilic group 2, and a hydrophobic group 3; and a conjugate 7 comprises a binding substance 1, a DNA 5a, and a hydrophilic molecule 6. The hydrophilic group 2 of each of the amphiphilic compounds 4 and the DNA 5a of each of the conjugates 7 are bound to the inner wall constituting the channel 23, that is, an inner surface of the channel 23, optionally via the binding substances 1. As described above, the binding substance 1 is not essential. Furthermore, it is not required that the entire inner side of the channel 23 be coated, and it is sufficient for at least a part of the inner side of the channel 23, specifically, at least the opening 31 to be coated.

FIG. 2(B) and FIG. 2(C) show a process of adhering the cell to the opening 31. In order to adhere the cell in a state shown in FIG. 2(A) to the opening 31, first, the above-described photoreactive substance (not shown) is provided to the opening 31. The photoreactive substance may be provided to the opening 31 in advance. Alternatively, the photoreactive substance may be provided to the opening 31 by introducing the photoreactive substance into the channel 23. The photoreactive substance is preferably bound to the DNA 5a. Thereafter, as shown in FIG. 2(B), the opening 31 is irradiated with light to excite the photoreactive substance. Active oxygen produced by the excitation of the photoreactive substance cleaves the DNA 5a, and thereby the hydrophilic molecule 6 that has been inhibiting a non-covalent bonding between the hydrophobic group 3 and a cell membrane of the cell C is dissociated from the inner wall of the channel 23. Since the remaining DNA fragment 5b is not big enough to inhibit the binding between the hydrophobic group 3 and the cell C, the hydrophobic group 3 and the cell membrane of the cell C non-covalently bond to each other, and thereby the cell C is adhered to the opening 31.

In the microchannel device 40 according to the present embodiment, a cell adhesion ability of the opening 31 can be expressed at an arbitrary timing. Accordingly, if a contaminant or a cell other than the target cell C is captured at the opening 31, it can be released from the opening 31 by reversing the pressure difference between the channel 23 and the channel 24. On the other hand, if the target cell C is captured at the opening 31, the cell C can be adhered to the opening 31 by irradiating the opening 31 with light. Once the cell C is adhered to the opening 31, it is not required to maintain the pressure difference between the channel 23 and the channel 24. Namely, according to the microchannel device 40 according to the present embodiment, cells can be selectively and easily captured and analyzed at an arbitrary timing by using light having an arbitrary wavelength.

Next, a method for adhering a cell onto a base material using the above-described cell adhesion composition will be described. The method for adhering a cell onto a base material according to one embodiment of the present invention comprises steps of: (a) coating the base material with the above-described cell adhesion composition; (b) bringing the above-described photoreactive substance into contact with the base material; (c) irradiating the base material with light to excite the photoreactive substance; and (d) bringing the cell into contact with the base material.

In step a, the above-described cell adhesion base material is obtained by coating the base material with the above-described cell adhesion composition.

The base material coated in step a is not particularly limited, and examples of materials and shapes and forms of the base material are as described above. Specific examples of the base material include a slide glass, culture dish, multi-well plate, inner wall of a microchannel of a microchannel device, and the like.

A coating method is not particularly limited, and for example, the base material may be coated by bringing the cell adhesion composition in a liquid form into contact with the base material. A method of bringing the cell adhesion composition into contact with the base material is not particularly limited, and for example, the cell adhesion composition may be added dropwise onto the base material, or the base material may be immersed in the cell adhesion composition.

In step b, the photoreactive substance is brought into contact with the base material. This step provides the photoreactive substance to the base material. The photoreactive substance is preferably bound to the DNA of the conjugate bound to the base material. Details of the photoreactive substance are as described above. Step b may be performed after step a or at the same time as step a. In other words, the photoreactive substance may be brought into contact with the base material coated with the cell adhesion composition, or the cell adhesion composition and the photoreactive substance may be brought into contact with the base material at the same time. In a case where the cell adhesion composition and the photoreactive substance are brought into contact with the base material at the same time, the photoreactive substance may be contained in the cell adhesion composition.

In step c, the base material is irradiated with light to excite the photoreactive substance. The excited photoreactive substance produces active oxygen, and the active oxygen cleaves the DNA of the conjugate. Accordingly, by this step, the hydrophilic molecule that has been inhibiting cell adhesion is dissociated from the conjugate, and thereby the hydrophobic group, which has the cell adhesion ability and has been hidden under the hydrophilic molecule, of the amphiphilic compound is exposed to the outermost part of the surface of the base material.

A wavelength of light, an irradiation intensity, and an irradiation time are not particularly limited as long as the photoreactive substance can be excited. A wavelength of light is preferably more than 380 nm from the viewpoint of preventing damage to cells. A wavelength of light may be, for example, 430 nm or more, 450 nm or more, or 480 nm or more. An irradiation time may be, for example, 1 second or longer, 10 seconds or longer, or 60 seconds or longer.

Step c may be performed after step b.

In step d, a cell is brought into contact with the base material. By this step, the hydrophobic group of the amphiphilic compound non-covalently bonds to the cell membrane, and thereby the cell is adhered to the base material. A method of bringing the cell into contact with the base material is not particularly limited, and for example, a cell suspension may be added dropwise onto the base material, or the base material may be immersed in the cell suspension. Step d may be performed at any stage after step a. In a case where step d is performed before step b, it is preferable that step b (bringing the photoreactive substance into contact) and the irradiation with light (step c) be performed while maintaining a state in which the cell is in contact with the base material. In a case where step d is performed at the same time as step b, or after step b and before step c, it is preferable that the irradiation with light (step c) be performed while maintaining a state in which the cell is in contact with the base material.

According to the method for adhering a cell onto a base material according to the present embodiment, cells can be adhered to the base material at an arbitrary timing and in a short irradiation time by using light having an arbitrary wavelength.

### EXAMPLES

(Preparation)

1. Preparation of PEG-DNA-BSA

20-mer DNA (sequence: TCTATCTGCAGGCGCTCTCC) having a carboxy group at the 5′-end and a thiol group at the 3′-end was synthesized. This DNA and BSA were respectively dissolved in 10 mM MOPS-KOH at pH 7.0, and thereby a DNA solution and a BSA solution were obtained. The BSA solution and the DNA solution were mixed at a molar ratio of 1:5. 1-(3-Dimethylaminopropyl)-3-ethylcarbodiimide (EDC) was mixed with this mixed solution such that a final concentration became 10 mM, and the carboxy group at the 5′-end of the DNA and an amino group of the BSA were bound. Excess DNAs were removed using a spin column. Thereafter, PEG-maleimide (weight-average molecular weight of PEG: 20000) was added together with 10 mM MOPS-KOH at pH 7.0 such that a molar ratio of BSA and PEG became 1:10, and they were mixed. DNA-BSA and PEG were bound by incubating the mixed solution for 30 minutes, and the reaction was stopped by mixing DTT at a final concentration of 1 mM.

2. Preparation of PEG-Lipid-BSA

BSA and PEG-lipid-NHS were respectively dissolved in 10 mM MOPS-KOH at pH 7.0, and thereby a BSA solution and a PEG-lipid solution were obtained. As the PEG-lipid-NHS, oleyl-O-polyethylene glycol-succinyl-N-hydroxy-succinimidyl ester (weight-average molecular weight of PEG: 2000, “SUNBRIGHT OE-020CS” manufactured by NOF CORPORATION) was used. The BSA solution and the PEG-lipid solution were mixed at a molar ratio of 1:10, and incubated for 30 minutes at room temperature. Tris-HCl at pH 6.8 was added to stop the reaction.

3. Preparation of PEG-Phospholipid-DNA-BSA

PEG-phospholipid-DNA-BSA was prepared in the same manner as in the preparation of PEG-DNA-BSA except that PEG-phospholipid-maleimide was used instead of PEG-maleimide. As the PEG-phospholipid-maleimide, N—[N′-(3′-maleimido-1′-oxopropyl)aminopropylpolyoxyethylene oxycarbonyl]-1,2-distearoyl-sn-glycero-3-phosphoethanolamine (weight-average molecular weight of PEG: 2000, “SUNBRIGHT DSPE-020MA” manufactured by NOF CORPORATION) was used.

### Example 1

PEG-DNA-BSA and PEG-lipid-BSA were mixed at a ratio of 1:5 so that a total concentration of BSA became 0.5 mg/mL This mixed solution was added dropwise onto a washed cover glass (24 mm×36 mm, t 0.17 mm), and thereby a substrate having a surface coated with PEG-DNA-BSA and PEG-lipid-BSA was obtained.

YOYO-1 (maximum absorption wavelength 491 nm, maximum fluorescence wavelength 509 nm) was added into a buffer such that a final concentration became 10 μM, and the mixture was added dropwise onto the substrate. Thereafter, a predetermined circular region was irradiated with excitation light for 10 seconds using a diaphragm to impart cell adhesive properties to the circular region. After the excitation, liberated PEG, the fluorescent dye, degraded DNAs, and the like were washed away with a buffer.

Cells were suspended in a medium not containing serum such that a concentration became 1×10⁵ cells/mL The cell suspension was brought into contact with the substrate, and after 10 minutes, excess cells were washed away with a medium containing serum. The cells on the substrate were cultured, and after one day, the cells on the substrate were observed using a phase-contrast microscope. The cells were adhered to and extended within the circular region and formed a circular pattern.

### Example 2

PEG-DNA-BSA and PEG-lipid-BSA were mixed at a ratio of 1:5 so that a total concentration of BSA became 0.5 mg/mL This mixed solution was introduced into the channel 23 of the microchannel device as shown in FIGS. 1(A) and 1(B) to coat the inside of the channel 23 with PEG-DNA-BSA and PEG-lipid-BSA.

Cells were suspended in phosphate buffered saline (PBS) such that a concentration became 1×10⁵ cells/mL The cell suspension was introduced into the channel 23, and PBS was introduced into the channel 24. A flow velocity was adjusted such that a pressure in the channel 23 became higher than a pressure in the channel 24, and the desired cell was held at the opening 31. In a case where cell debris or cells other than the desired cell were captured at the opening 31, a pressure difference was reversed to release them from the opening 31.

After the desired cell was captured at the opening 31, PBS containing YOYO-1 was introduced into the channel 23. The opening 31 was irradiated with excitation light for 10 seconds. After the irradiation, PBS was introduced into the channel 23 to wash away liberated PEG, the fluorescent dye, degraded DNAs, and the like. The desired cell was found adhered to the opening 31.

### Example 3

PEG-phospholipid-DNA-BSA was suspended in a buffer such that a concentration of BSA became 0.5 mg/mL This suspension was added dropwise onto a washed cover glass (24 mm×36 mm, t 0.17 mm), and thereby a substrate having a surface coated with PEG-phospholipid-DNA-BSA was obtained.

Cells were suspended in a medium not containing serum such that a concentration became 1×10⁵ cells/mL The cell suspension was brought into contact with the above-described substrate. After confirming adhesion of the cells to the substrate, the substrate was washed with a medium containing serum, and the cells were cultured until they became confluent.

YOYO-1 was added into the medium such that a final concentration became 10 μM, and the mixture was added dropwise onto the substrate. Thereafter, a predetermined circular region was irradiated with excitation light for 10 seconds using a diaphragm. Cells in the circular region dissociated from the substrate and floated in the medium. The floating cells in the medium were recovered.

### REFERENCE SIGNS LIST

1 Binding substance
2 Hydrophilic group
3 Hydrophobic group
4 Amphiphilic compound
5a DNA
5b DNA fragment
6 Hydrophilic molecule
7 Conjugate
21 Cover glass
22 Substrate
23, 24 Channel
25, 26, 27 Inlet
28, 29 Outlet
40 Microchannel device
30 Communicating portion
31 Opening
32 Hole
C Cell

1: A cell adhesion composition comprising:
an amphiphilic compound; and
a conjugate of a DNA and a hydrophilic molecule,
wherein the amphiphilic compound has a hydrophobic group that can non-covalently bond to a cell membrane, and a hydrophilic group, and
wherein a weight-average molecular weight of the hydrophilic molecule of the conjugate is larger than a weight-average molecular weight of a hydrophilic molecule from which the hydrophilic group of the amphiphilic compound derives.

2: The composition according to claim 1,
wherein the hydrophilic group is a residue of a hydrophilic molecule selected from the group consisting of polyalkylene glycol, polyglycerin, polysaccharide, polylactic acid, polyvinyl alcohol, polyacrylic acid, and polyacrylamide, and
the hydrophobic group is an aliphatic hydrocarbon group having 7 to 22 carbon atoms, or a residue of a phospholipid having an aliphatic hydrocarbon group having 7 to 22 carbon atoms.

3: The composition according to claim 1, wherein the hydrophilic molecule of the conjugate is a hydrophilic molecule selected from the group consisting of polyalkylene glycol, polyglycerin, polysaccharide, polylactic acid, polyvinyl alcohol, polyacrylic acid, and polyacrylamide.

4: The composition according to claim 1, wherein the hydrophilic group is a residue of polyethylene glycol, and the hydrophobic group is an aliphatic hydrocarbon group having 10 to 20 carbon atoms, or a residue of a phospholipid having an aliphatic hydrocarbon group having 10 to 20 carbon atoms.

5: The composition according to claim 1, comprising one or more conjugates per molecule of the amphiphilic compound.

6: The composition according to claim 1, wherein the weight-average molecular weight of the hydrophilic molecule of the conjugate is more than 1 time the weight-average molecular weight of the hydrophilic molecule from which the hydrophilic group of the amphiphilic compound derives.

7: A cell adhesion base material comprising:
a base material;
one or more amphiphilic compounds; and
one or more conjugates of a DNA and a hydrophilic molecule,
wherein each of the amphiphilic compounds has a hydrophobic group that can non-covalently bond to a cell membrane, and a hydrophilic group,
the hydrophilic group of each of the amphiphilic compounds and the DNA of each of the conjugates are bound to the base material, and
a weight-average molecular weight of the hydrophilic molecule of the conjugate is larger than a weight-average molecular weight of a hydrophilic molecule from which the hydrophilic group of the amphiphilic compound derives.

8: The cell adhesion base material according to claim 7, comprising one or more conjugates per molecule of the amphiphilic compound.

9: A cell adhesion base material comprising:
a base material; and
one or more conjugates of an amphiphilic compound and a DNA,
wherein each of amphiphilic compounds has a hydrophobic group that can non-covalently bond to a cell membrane, and a hydrophilic group bound to the DNA, and
the DNA is bound to the base material.

10: The cell adhesion base material according to claim 7, further comprising a photoreactive substance that produces active oxygen upon light irradiation.

11: A microchannel device comprising a channel in which at least a part of an inner side is coated with the cell adhesion composition according to claim 1.

12: The microchannel device according to claim 11, comprising:
a first channel;
a second channel adjacent to the first channel; and
a communicating portion that connects the first channel to the second channel and has an opening on the side of the first channel in which a cell can be captured,
wherein the part of the inner side that is coated with the cell adhesion composition includes an inner side of the first channel.

13: A method for adhering a cell onto a base material, the method comprising:
coating the base material with the cell adhesion composition according to claim 1;
bringing a photoreactive substance that produces active oxygen upon light irradiation into contact with the base material;
irradiating the base material with light to excite the photoreactive substance; and
bringing the cell into contact with the base material.

14: The cell adhesion base material according to claim 8, further comprising a photoreactive substance that produces active oxygen upon light irradiation.

15: The cell adhesion base material according to claim 9, further comprising a photoreactive substance that produces active oxygen upon light irradiation.

16: A method for adhering a cell onto a base material, the method comprising:
coating the base material with the cell adhesion composition according to claim 2;
bringing a photoreactive substance that produces active oxygen upon light irradiation into contact with the base material;
irradiating the base material with light to excite the photoreactive substance; and
bringing the cell into contact with the base material.

17: A method for adhering a cell onto a base material, the method comprising:
coating the base material with the cell adhesion composition according to claim 3;
bringing a photoreactive substance that produces active oxygen upon light irradiation into contact with the base material;
irradiating the base material with light to excite the photoreactive substance; and
bringing the cell into contact with the base material.

18: A method for adhering a cell onto a base material, the method comprising:
coating the base material with the cell adhesion composition according to claim 4;
bringing a photoreactive substance that produces active oxygen upon light irradiation into contact with the base material;
irradiating the base material with light to excite the photoreactive substance; and
bringing the cell into contact with the base material.

19: A method for adhering a cell onto a base material, the method comprising:
coating the base material with the cell adhesion composition according to claim 5;
bringing a photoreactive substance that produces active oxygen upon light irradiation into contact with the base material;
irradiating the base material with light to excite the photoreactive substance; and
bringing the cell into contact with the base material.

20: A method for adhering a cell onto a base material, the method comprising:
coating the base material with the cell adhesion composition according to claim 6;
bringing a photoreactive substance that produces active oxygen upon light irradiation into contact with the base material;
irradiating the base material with light to excite the photoreactive substance; and
bringing the cell into contact with the base material.
